(19) United States
(12) Patent Application Publication
(10) Pub. No.: US 2011/0137047 A1
(43) Pub. Date: Jun. 9, 2011

(54) Process for enantiomerically pure 8-Aryloctanoic acids as Aliskiren

(75) Inventor: Milan Soukup, Sarasota, FL (US)

(73) Assignee: CarboDesign LLC, Sarasota, FL (US)

(21) Appl. No.: 12/925,151

(22) Filed: Oct. 15, 2010

Related U.S. Application Data
(60) Provisional application No. 61/283,616, filed on Dec. 7, 2009.

(51) Int. Cl. C07C 231/02; C07D 405/04; C07D 203/08; C07D 307/33; C07C 57/13; C07C 237/22; C07C 231/12; C07C 11/28

(52) U.S. Cl. 548/517; 548/968; 549/321; 562/595; 564/134; 564/158; 585/16

(57) ABSTRACT

The present invention relates to a novel manufacturing process and novel intermediates useful in the synthesis of pharmaceutically active compounds, especially rennin inhibitors such as Aliskiren. The invention describes a preparation of enantiomerically pure 8-aryloctanoic acids of general formula I from readily available key intermediate, chiral cis-diacid of formula II, aziridine of formula XI and a monocyclic compound of formula III.

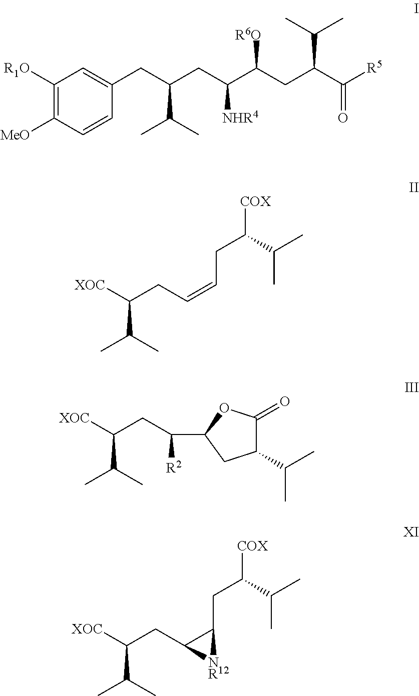

This application claims priority to U.S. Provisional Application Ser. No. 61/283,616 filled Dec. 7, 2009.

### BACKGROUND OF THE INVENTION

8-Aryloctanoic acids of a general formula I, having the 2S,4S,5S,7S-configuration,

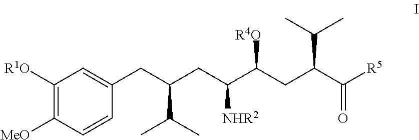

especially compound such as Aliskiren, wherein R¹ represents CH₃OCH₂CH₂CH₂—, R² and R⁴ hydrogen and R⁵—NHCH₂C(CH₃)₂CONH₂ (INN name: 5-amino-N-(2-carbamoyl-2-methylpropyl)-4-hydroxy-2-isopropyl-7-[4-methoxy-3-(3-methoxypropoxy)benzyl]-8-methyl-nanoamide), are excellent new antihypertensive which interfere with the rennin-angiotensin system.

After discovery of biological activity of these compounds of general formula I in 1994, first synthesis of Aliskiren has been disclosed (U.S. Pat. No. 5,559,111 and EP 0 678 503). Since Aliskiren contains 4 chiral centers, synthesis of enantiomerically pure compound is very complex. After 2001 many patents and publications have been filed or published claiming alternative routes to Aliskiren (WO 01/09083, WO 01/09079, EP 1 215 201, WO 02/02508, WO 02/02500, WO 02/02487, WO 02/08172, WO 02/092828, WO 02/02500, WO 03/103653, UK 2 431 640, GB 2 431 641, GB 2 431 642, GB 2 431 643, GB 2 431 644, GB 2 431 645, GB 2 431 646, GB 2 431 647, GB 2 431 48, GB 2 431 649, GB 2 431 650, GB 2 431 651, GB 2 431 652, GB 2 431 653, GB 2 431 654, WO 2005/054177, WO 2005/090305, WO 2005/051895, WO 2006/131304, WO2006/095020, WO2006/024501, WO2007/054254, WO2007/039183, EP 2 062 874, EP 1958 666, WO 2007/006532, WO2007/045420, WO2008/155338, WO2008/119804, CA 2 634 513, WO2007/048620, WO2007/118681, US2009/0076062, WO2010/010165, EP2189442, WO2009/049837, Tetrahedron Letters 2000, 41, 10085, ibid. 2000, 41, 10091, ibid. 2001, 42, 4819, Drugs Fut. 2001, 1139, J. Org. Chem. 2002, 67, 4261, Helv. Chim Acta 2003, 86, 2848, Tetrahedron Letters 2005, 46, 6337, J. Org. Chem. 2006, 71, 4766, Organic Process & Develop 2007, 11, 584, Tetrahedron Letters 2008, 49, 5980 and Org. Lett. 2010, 12, 1816). Nevertheless, none of them fulfill requirements for a short and cost effective manufacturing process.

As recently claimed in WO2007/045420 and other patents, trans-configurated (2S,7S)-2,7-diisopropyloct-4-enedioic acid or derivatives thereof have been used as a starting material in the synthesis of compounds of formula I. In the aliphatic chain C(5)-amino and C(4)-hydroxyl groups have been introduced via a three step reaction sequence, starting with halo lactonization of trans-double bond, then displacement of the halogen with azide followed by reduction or hydrogenation of the azide group. In order to introduce both N- and O-functions with the desired stereochemistry, as defined in compounds of formula I, according to this concept only trans-configurated double bond can be used.

### SUMMARY OF THE INVENTION

The present invention discloses a novel efficient process for the manufacture of enantiomerically pure compounds of general formula I, specifically of Aliskiren, as shown in Scheme 1:

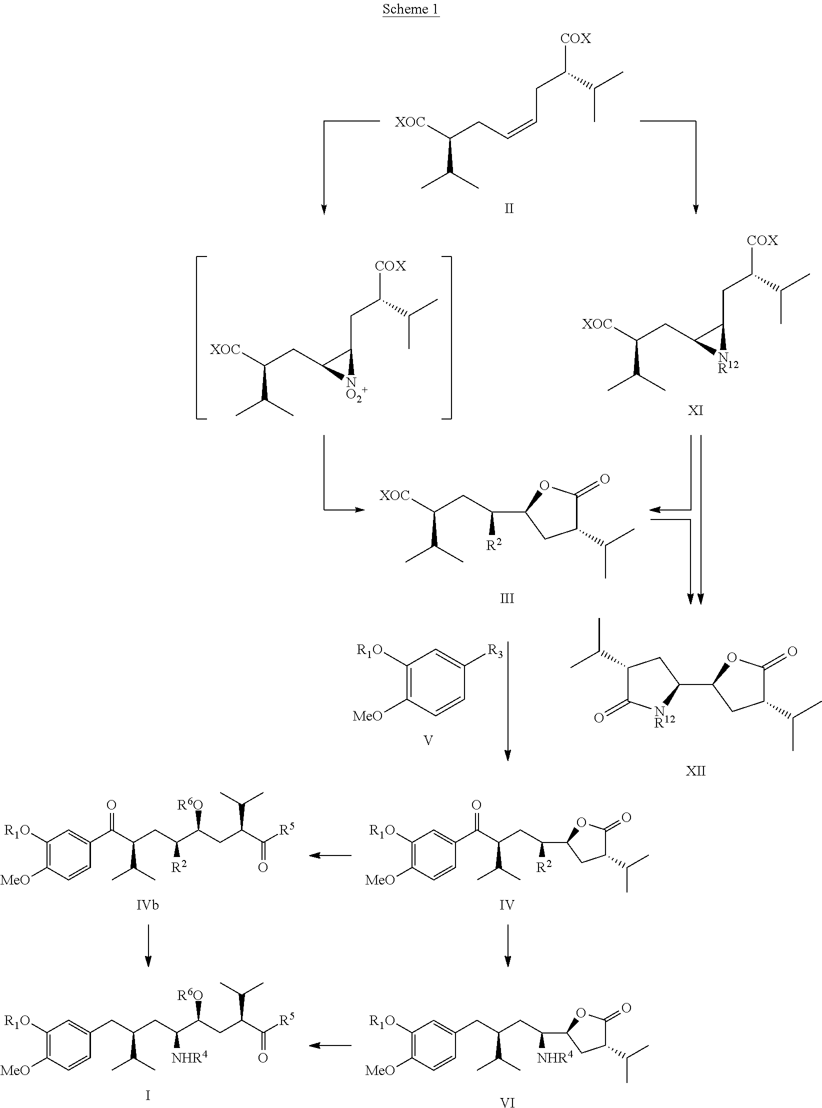

It has been unexpectedly found that compound of formula I and intermediates thereof (formulas III, IV, VI and XII) containing 4 chiral centers, can be efficiently prepared by a simple sequence of steps starting from an inexpensive chiral compound of a general formula II which possesses only two chiral centers and specifically, cis-configurated double bond. Until now potential of this cis-configurated double bond, in connection with alternative methods for introduction of C(5)-amino and C(4)-hydroxy functions, has not been considered: As unexpectedly found nitrogen function at C(5)-atom and oxygen function at C(4)-atom can be very efficiently formed via either nitration or “aziridination” of this cis-configurated double bond in the compound of formula II. Either nitro lactonization, preferably with AcONO₂, or amino lactonization with in situ generated nitrene, of the cis-configurated double bond occurs always stereoselectivly initially as a cis-addition. Subsequent intramolecular opening of nitronium intermediate leads then exclusively to trans-3,5-disubstituted 5-membered lactone of formula III. Similar the aziridine of formula XI can also be selectively opened in antarafacial way providing compound of formula III or XII, both very important intermediates in synthesis of Aliskiren. As shown in Scheme 1 according to both approaches the new chiral centers at C(4) and C(5) atoms are formed with high stereo selectivity.

Chiral compound of formula II is accessible from inexpensive starting materials as briefly shown in Scheme 2:

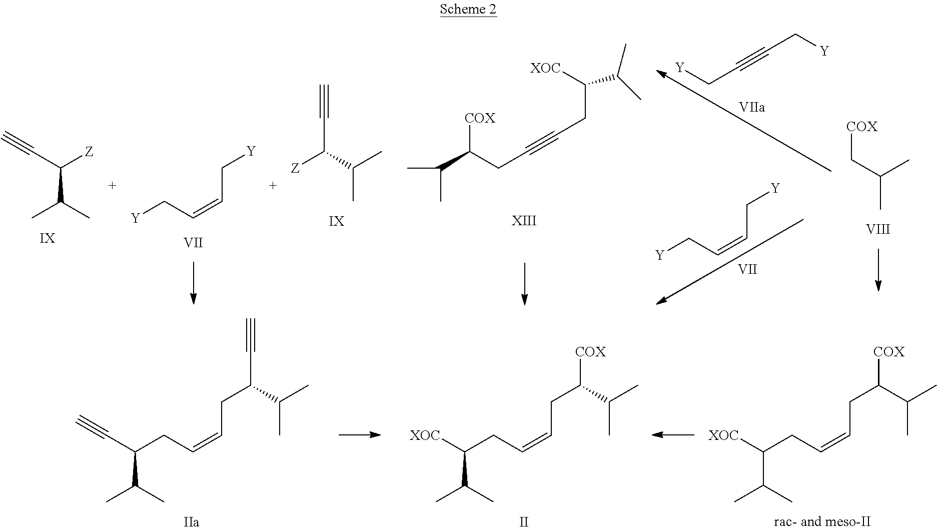

The invented process for enantiomerically pure compounds as shown in Scheme 1 can also be applied for preparation of racemic compounds of formulas III, IV, IVb, VI and XII which can be subjected at any stage of the synthesis to a resolution step providing the enantiomerically pure compounds.

### DETAILED DESCRIPTION OF THE INVENTION

The present invention (Scheme 1) claims a process for the preparation of a compound of the general formula I

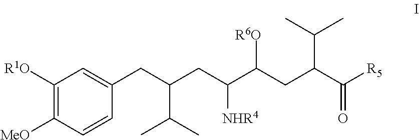

wherein R¹ represents hydrogen, linear or brunched C₁₋₆ alkyl, C₁₋₆ alkoxy-C₁₋₆ alkyl, aryl, alkylaryl, arylalkyl, carbamoyl, trifluoracetyl, mesyl, tosyl, trifluoromethanesulfonyl, trialkylsilyl, preferably CH₃OCH₂CH₂CH₂—, acyl, formyl, R⁴ represents hydrogen, alkyl, aryl, alkylaryl, arylalkyl, hydroxy, alkoxy, aryloxy, arylalkyloxy, alkylaryloxy, trialkylsilyl, trialkylsilyloxy, with heteroatom(s) substituted alkyl, aryl, alkylaryl, arylalkyl, preferably benzyl, mono-, di- or tri-methoxybenzyl, or N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —(O)C—Oalkylaryl, —C(O)Oarylalkyl, preferably —C(O)Obenzyl (Cbz) or —C(O)Otert.-butyl (BOC),
R⁵ is hydroxy, linear or brunched C₁₋₆ alkyloxy, aryloxy, alkylaryloxy, arylalkyloxy, trialkylsilyloxy, halogen, preferably chlorine or bromine, —NH₂, —NMe₂ or —NHCH₂C(CH₃)₂CONH₂ and
R⁶ represents hydrogen, alkyl, aryl, alkylaryl, trialkylsilyl, alkylarylsilyl, with heteroatom(s) substituted alkyl, aryl, alkylaryl, arylalkyl, preferably benzyl, mono-, di- or tri-methoxybenzyl, or other O-protective group, in particular one which together with O forms an ester or carbonate, as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably —C(O)Obenzyl (Cbz), —C(O)Otert.-butyl (BOC), formyl or acetyl;
comprising following steps:
a) reaction of the compound of formula II,

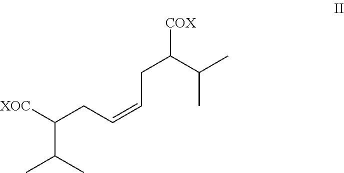

wherein X represents —OH, linear or brunched C₁₋₆-alkyloxy, aryloxy, alkylaryloxy, arylalkyloxy, trialkylsilyloxy or alkylarylsilyloxy, halogen, preferably chlorine or bromine,
—OC(O)R⁷ or —OC(O)OR⁷, wherein R⁷ is linear or brunched C₁₋₆-alkyl, arylalkyl, preferably methyl, ethyl, tert.-butyl or benzyl,
—NR⁸R⁹, wherein R⁸ and R⁹ are independently from each other hydrogen, lower alkyl, arylalkyl, preferably —NH₂, —NMe₂ or dibenzyl, or in particular R⁸ and R⁹ can form together with N a 5- or 6-membered heterocyclic ring which may contain one or more heteroatoms selected from N or O and, which can be unsubstituted or substituted, preferably such as 4-alkyl-oxazolidin-2-one-3-yl containing also a chiral center such as e.g. 4(R)- or 4(S)-benzyl-oxazolidin-2-one-3-yl,
—NR¹⁰R¹¹, wherein R¹⁰ and R¹¹ are independently from each other lower alkyl, arylalkyl or in particular R¹⁰ and R¹¹ can form together with N and O a 5- or 6-membered heterocyclic ring which may contain one or more heteroatoms selected from N or O and, which can be unsubstituted or substituted, containing also a chiral center, preferably —NMeOMe and
wherein the double bond is specifically cis-configurated,
with a nitration agent containing NO₂⁺-agent, preferably nitration agent defined such as NO₂-Lvg, wherein Lvg is a leaving group, preferably NO₂OAc or NO₂BF₄ or Cerium ammonium nitrate (CAN), providing compound of formula III

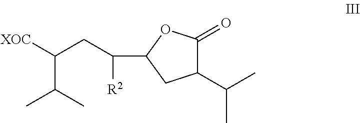

wherein X is the same as defined for X in the compound of formula II and R² represents NO₂;
b) reaction of the compound of formula III with a compound of formula V,

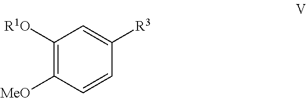

wherein R¹ is the same as defined for the compound of formula I and R³ is a metal containing group such as Li, Na, Mghalide (Grignard), —Znhalide, Mnhalide, cuprate —Cuhalide, —Cehalides, boronic acid as —B(OH)₂, preferably —Li or —MgBr or —Mg ate complex, providing compound of formula either IV or IVa,

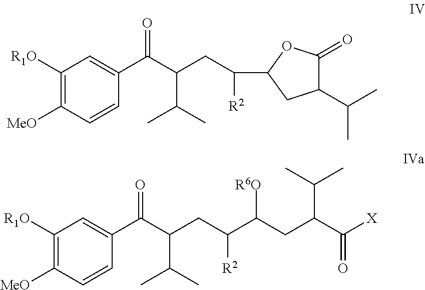

wherein R¹ is the same as defined for the compound of formula I,
R² is NO2 and
R⁶ is hydrogen, alkyl, aryl, alkylaryl, trialkylsilyl, alkylarylsilyl, with heteroatom(s) substituted alkyl, aryl, alkylaryl, arylalkyl, preferably mono-, di- or tri-methoxybenzyl, or other O-protective group, in particular one which together with 0 forms an ester or carbonate, as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably formyl, acetyl, —C(O)Obenzyl (Cbz) or —C(O)O-tert.-butyl (BOC);
c) reaction of
   i) either the compound of formula IV directly with R⁵—H, wherein R⁵ is the same as defined for the compound of formula I,
      preferably —NHCH₂C(CH₃)₂CONH₂
   ii) or the compound of formula IVa via a coupling step with a peptide coupling reagent followed by reaction with R⁵—H, wherein R⁵ is the same as defined for the compound of formula I, preferably —NHCH₂C(CH₃)₂CONH₂,
   providing a compound of formula IVb,

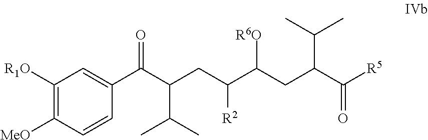

wherein R¹, R⁵ and R⁶ are the same as, defined for the compound of formula I and R² is NO₂;
d) reduction and/or hydrogenation of C(8)-oxo and C(5)-nitro groups in the compound of formula IVb, either simultaneously or in separate steps, followed by subsequent protection or removal of protective group(s) according to as they are defined in the compound of formula I.

The present invention claims also an alternative route in which compounds of formula IV, or alternatively IVa,

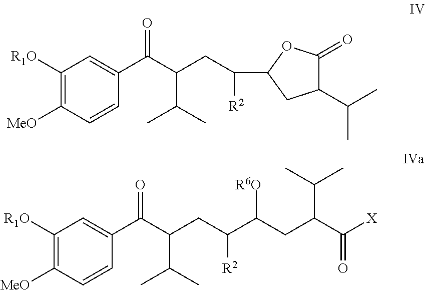

are first reduced with a reducing agent or hydrogenated. During this process C(8)-oxo and C(5)-nitro groups are, simultaneously or in separate steps, reduced. After appropriate protection of C(5)-amino group, compounds of formula VI or VIa can be obtained,

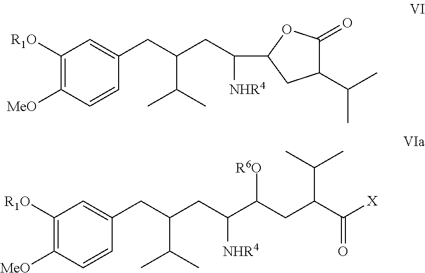

wherein R¹, R⁴, R⁶ are the same as defined for the compound of formula I and X is the same as defined for compound of formula II. In subsequent step compounds of formula VI or Vla are then subjected reaction with R⁵—H, wherein R⁵ is the same as defined for the compound of formula I, preferably NH₂CH₂C(CH₃)₂CONH₂: Either the lactone of formula VI can be opened directly with R⁵—H or compound of formula VIa, after prior protection of C(4)-hydroxy group, subjected to a coupling reaction with R⁵—H as already reported in e.g. U.S. Pat. No. 5,559,111 from Sep. 24, 1996.

When referring to compounds described in the present invention, it is understood that references are also being made to salts thereof.

Depending on the choice of starting materials the compounds can be present in the form of one possible isomer or a mixture of stereoisomers thereof, for example as enantiomerically pure compound or as isomer mixtures, such as racemates, diastereomer mixtures etc., depending on the number of asymmetric carbon atoms.

In this invention racemic compounds of formulas II, III or IIIa, IV or IVa or IVb and VI can be subjected at any stage of the synthesis to a resolution or separation step using (chiral) agent or including an enzymatic step or another separation method known as e.g. preparative HPLC or SMB etc. As the resolution agent any chiral acid or base as commonly used for resolution of nitrogen- or alcohol- or carboxylate-containing compounds, can be used.

In this invention a characteristic of protective groups is that they can be removed readily (without the occurrence of undesired secondary reactions) for example by solvolysis, reduction, or alternatively under physiological conditions (as e.g. enzymatic cleavage or formation). Different protective groups can be selected so that they can be removed selectively at different stages of the synthesis while other protective groups remain intact. The corresponding alternatives can be selected readily by a person skilled in the art from those given in the standard reference works mentioned in literature (as e.g. Mc Omie “Protective Groups in Organic Chemistry” or Green et al. “Protective Groups in Organic Synthesis”) or in the description or in the claims or the Examples.

In a preferred embodiment of the invention, preparation of enantiomerically pure compound of general formula I having the formula as given,

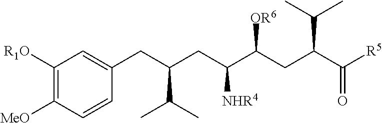

wherein R¹, R⁴, R⁵ and R⁶ are the same as defined above for the compound of formula I and the compounds of formula II, III, IIIa, IV, IVa, IVb and VI have the configuration as defined in Scheme 1, can be carried out:

In the nitration step, nitrogen function at the C(5)-atom and oxygen function at C(4)-atom are introduced simultaneously occurring formally as trans-addition to cis-configurated double bond: After initial cis-addition of NO₂-cation to the double bond, in situ formed nitronium-cation, is spontaneously opened via an intramolecular mechanism leading exclusively to compound of formula III which can react further to compound of formula IIIa dependent on reaction conditions and reagent employed.

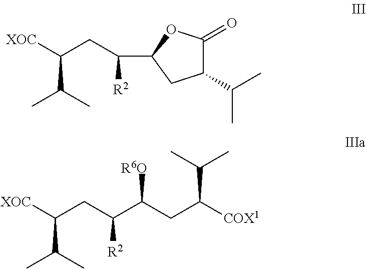

It was unexpectedly found that the lactone formation can occur with high diastereoselectivity providing exclusively 5-membered either trans- or cis-3,5-disubstituted lactone of formula III dependent on the functional group X employed in the nitration process as reported in analogy for iodolactonization in J. Org. Chem. 1989, 54, 1178 and Liebigs Ann. Chem. 1990, 323.

The “nitration” agent containing NO₂⁺-source can be any agent defined as NO₂-Lvg, wherein Lvg is a leaving group. As reported in literature many reagents fulfill these requirements and can be used (J. Prakt. Chem. 1991, 333, 677) as for example: NO₂OAc prepared in Ac₂O and AcOH (J. Org. Chem. 1977, 42, 2939, ibid. 1963, 28, 1765 and Chem. Ber. 1958, 745),

NO₂BF₄ in the presence of LiClO₄ in methylenechloride, acetonitrile, or ethylacetate (J. Org. Chem. 1982, 47, 3679, ibid. 1971, 36, 3641,
cerium(IV) ammonium nitrate (CAN) in Ac₂O, methylenechloride or acetonitrile or CAN in the presence of NaNO₂ (J. Chem. Res. (S) 2003, 497, Bull. Inst. Chem. Res. Kyoto Univ. 1989, 67, 112, Tetrahedron 2004, 60, 397,
fuming nitric acid in conc. sulfuric acid or fluorosulfonic acid or trifluoroacetic acid (J. Heterocyclic Chem. 1984, 21, 725,
NH₄NO₃ and TfOH or HBF₄ in methylenechloride (Tetrahedron Letters 1986, 27, 873, NH₄NO₃ in trifluoroacetic anhydride in acetonitrile (J. Org. Chem. 1986, 51, 2617,
dinitrogen tetroxide in the presence of oxygen (J. Am. Chem. Soc. 1958, 80, 338, Org. Synthesis Vol. 50, 84, J. Am. Chem. Soc. 1967, 89, 3005, J. Org. Chem. 1997, 62, 6498,
sodium nitrite with iodine in ethyl acetate (Chem. Lett. 1986, 1747 and nitric oxide (Chem. Lett. 1995, 505).

In the preferred embodiment of this invention as shown in Scheme 1 the enantiomerically pure compound of formula II, wherein X represents —OH, —OMe, —NMe₂ or Evans chiral auxiliary (such as 4(S)-benzyl-oxazolidin-2-one-3-yl) is converted with either NO₂OAc in Ac₂O and AcOH or with NO₂BF₄ or CAN in methylenechloride into the compound of formula III.

The compound of formula IIa (Scheme 2) can also be subjected directly to nitration step under similar conditions as used above for compound of formula II. During this nitration step and subsequent work-up the triple bond is oxidized and hydrolyzed to carboxylic acid, the double bond nitrated as discussed leading to the compound of formula III.

The compound of formula V, wherein R³ is metallic, especially an alkali or earth alkali metallic radical, as e.g. lithium, sodium, potassium or a group of the formula Mg-halogen, —Znhalogen, —Cer(halogen)₂ or boronic acid as —B(OH)₂, preferably —Li or —MgBr or —Mg ate complex, is prepared from the corresponding aromatic halide (a compound of formula V, wherein R³ is a halide, preferably bromide) and is used in situ in an inert solvent, such as THF etc. at a temperature range of −78° C. to 0° C. similar as reported in Novatis patent (p. 30, 78 and 82 in U.S. Pat. No. 5,559,111 from Sep. 24, 1996 or on page 24 and 25 in WO2007/045420 or J. Org. Chem. 2001, 66, 4333). To achieve sufficient selectivity in Grignard reaction the functional group X in the compounds of formula III or IIIa must be sufficiently activated as e.g. acid halide (X is preferably chlorine or bromine) or as Weinreb amide (X is preferably —NMeOMe) or as mixed anhydride (X is preferably OCOtert.-butyl etc.).

In the further embodiment of this invention instead of the above described reaction of organometallic compound of formula V with compound of formula III, polarity of both reaction components can be reversed: The compound of formula V, wherein R³ is hydrogen and R¹ is COCF₃, Mesyl, Tosyl or —SO₂CF₃ (Tetrahedon Letters 2002, 43, 7077), can be reacted with compound of formula III or IIIa, wherein X is halogen, preferably chlorine or bromine, or a mixed anhydride such as —OC(O)lower alkyl or —OC(O)Olower alkyl, preferably —OCOCF₃ or —OMesyl or —OSO2CF₃, under standard Friedel-Crafts conditions in the presence of catalyst used for Friedel-Crafts reaction as e.g. bortrifluoro etherate, aluminium chloride, metal halide, preferably Al-, Zn-, lanthanide- and Bi-halides (Tetrahedron Letters 2003, 44, 2937, ibid. 2003, 44, 5343, Tetrahedron 2004, 60, 10843). As solvent aprotic organic solvent, preferably chlorinated hydrocarbons as methylenechloride or aliphatic hydrocarbons as hexane or heptane can be used. After Fridel-Crafts reaction as shown in Scheme 1 the protecting/activating group R¹ is removed at any stage of the synthesis and replaced with any group as defined for the compounds of formula IV or IVa or IVb, VI or I.

The reduction or/and hydrogenation of 8-oxo group and 5-nitro groups in compounds of formula IV, IVa or IVb can be achieved either simultaneously or in separate steps. The preferred reduction method is hydrogenation in the presence of homogeneous or heterogeneous hydrogenation catalysts or reduction with metal hydrides, preferably sodium or lithium borohydride or trialkylsilanes in the presence of acid, preferably triethylsilane in the presence of triflic or trifluoroacetic acid or Lewis acid as bortrifluoro etherate, ZnCl₂, AlCl₃ or TiCl₄ at reaction temperature between −78 C until reflux.

The reaction of the compounds of formula IV or IVa and VI or Vla with a compound R⁵—H, preferably NH₂CH₂C(CH₃)₂CONH₂, can be carried out in many different ways:
i) comprising either initial opening of the 5-membered lactone ring, eventual protection of 4-hydroxy and/or 5-amino group(s) and reaction of free carboxylic acid, or ester thereof, with R⁵—H as known for preparation of ester or amides
ii) or alternatively by direct reaction of the lactone of formula IV or VI with R⁵—H as reported in Novartis patent (p. 22, 23, 24, 31, 32 in U.S. Pat. No. 5,559,111 from Sep. 24, 1996).

In the preferred embodiment of this invention, the lactones of formula IV or VI are directly reacted with NH₂CH₂C(CH₃)₂CONH₂ as reported in EP-A-678 503 (p. 124, 130 and 131) or WO02/02508 (example H1 p. 35, preparation of J1) or U.S. Pat. No. 5,559,111 (example 83).

Coupling with free carboxylic acid can be carried out according to standard peptide coupling method as also described for this step in U.S. Pat. No. 5,559,111 on page 22-25 or, as reported in analogues cases in Houben-Weyl, Methoden der organischen Chemie, 4^{th} Edition, Synthese von Peptiden1, Volume 15/II 1974, Volume IX, 1955, Volume E 11, 1985, Gerge Thieme Verlag, Stuttgart, The Peptides, (e. Gross and J. Meienhofer)

Volume 1 and 2, Academic Press, London 1979/1980 or M. Bodansky Principels of Peptide Synthesis, Springer Verlag, Berlin 1984. The condensation of free carboxylic acid with amine can be carried out in the presence of one of the coupling agents as e.g. DCC or other dialkyl carbodiimides, carbonyldiimidazole, 1,2-oxazolinium compounds, e.g. 2-ethyl-5-phenyl-1,2-oxazolium-3″-suphonate and 2-tert.-butyl-5-methylisoxazolium perchlorate, or a suitable acylamino compound, e.g. 2-ethoxy-1-ethoxy-carbonyl-1,2-dihydroquinoline, or activated phosphoric acid derivatives, bis(2-oxo-3-oxazolidinyl)phosphinic acid chloride or 1-benzotriazolyloxy-tris(dimethylamino)phosphonium hexafluorophosphate etc.

As further embodiment of the invention the compound of formula III (Scheme 1) can be subjected a reduction and/or hydrogenation, either in one or in several reaction steps, providing known bicyclic compound of formula XII which has been already used in the synthesis of compound of formula I (Aliskiren) as reported in U.S. Patent Appl. 61/279,995 from Oct. 29, 2009, WO 2007/045420 from Apr. 26, 2007, WO 2008/119804 from Oct. 9, 2008 and WO 2008/155338 from Dec. 24, 2008)).

As a preferred embodiment of the invention (Scheme 1), the compound of formula II

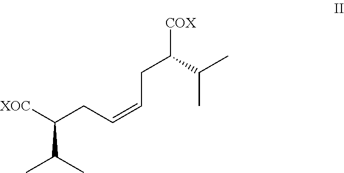

is reacted with a nitrene, prepared in situ
a) either by thermal decomposition of alkyl- or aryl- or arylakyl-azide, preferably benzyl azide, Mesyl-, Tosyl-azide, or alkyl azidoformiate such as N₃COOMe or N₃COObenzyl or N₃COOtert.-butyl, or acyl azide such as AcN₃ or PhCON₃ (Tetrahedron 1990, 46, 1911 or Tetrahedron Letters 1964, 52, 3953 or Can. J. Chem. 1968, 46, 3333)
b) or by base catalyzed decomposition of alkane or arene sulfonyl oxycarbamate such as alkyl- or benzyl- or tert.-butyl-OC(O)NHMesyl or -Tosyl or —Nosyl or H₂NSO₃CH₂CCl₃ in the presence of PhI(OAc)₂ and Rh-catalyst (Can. J. Chem. 1971, 49, 2610 or Tetrahedron Lett. 2009, 50, 3329 or Angew. Chem. Int. Ed. 2008, 47, 8703 or J. Org. Chem. 2005, 70, 3296 or J. Amer. Chem. Soc. 2002, 124, 13672) in inert organic solvent, preferably methylenechloride, THF under room or elevated temperature,
   providing a compound of formula XI,

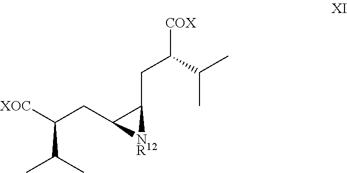

wherein X is the same as defined for compound of formula II and R¹² is hydrogen, alkyl, aryl, alkylaryl, arylalkyl, trialkylsilyl, hydroxy, alkoxy, arylalkyloxy, with heteroatom(s) substituted alkyl, aryl, alkylaryl, arylalkyl, preferably benzyl, mono-, di- or tri-methoxybenzyl, or other N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —(O)COalkylaryl, —C(O)Oarylalkyl, preferably formyl, acetyl, trifluoroacetyl, —C(O)Obenzyl (Cbz) or —C(O)Otert.-butyl (BOC), -Mesyl, -Tosyl or —SO₃CH₂CCl₃.

Since the starting compound of formula II possesses C₂-symmetry only one stereoisomer of formula XI is formed.

Acid or based or metal catalyzed rearrangement of the aziridine of formula XI, in analogy as reported in Tetrahedron Letters 1994, 35(24), 4073 and Org. Letters 2010, 12, 1816, leads, dependent on reaction conditions, to either compound of formula III or compound of formula XII, wherein X is the same as defined for compound of formula II, R₂ is NR⁴R¹² wherein R⁴ and R¹² are independently from each other arylalkyl, preferably benzyl, mono-, di- or trimethoxybenzyl, or trialkylsilyl or other N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably formyl, acetyl, trifluoroacetyl, —C(O)Obenzyl (Cbz) or —C(O)Otert.-butyl (BOC), or Mesyl and Tosyl, and

R¹² is hydrogen, lower alkyl, alkylaryl, arylalkyl, trialkylsilyl, —OH, —Oalkyl, —Oaryl, —Oalkylaryl, —Oarylalkyl, —Otrialkylsilyl, with heteroatom(s) substituted-alkyl, aryl, alkylaryl, arylalkyl, preferably methoxy, benzyl, mono-, di- or tri-methoxybenzyl, or other N-protective group, in particular one which together with N forms an amide or carbamate, as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably —C(O)Obenzyl (Cbz) or —C(O)Otert.-butyl (BOC), preferably formyl, acetyl, Mesyl, Tosyl or —SO₃CH₂CCl₃, dependent on the nitrene-reagent used and/or final de- or re-protection of aziridine nitrogen atom.

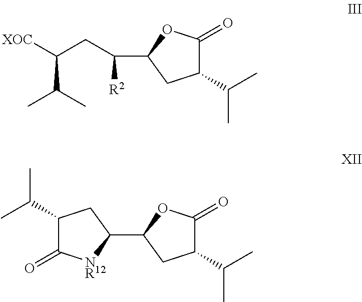

Since trans-3,5-disubtituated 5-membered lactone is more favored, opening of the aziridine of formula XI proceeds with high stereoselectivity control according to carboxylic acid opens in antarafacial way the aziridine ring, which results in inversion at the carbon atom bearing the oxygen function and retention at the carbon atom bearing the nitrogen function, leading to exclusively, trans-3,5-disubstituted lactone of formula III. Compound of formula XII can be formed from aziridine of formula XI directly or via compound of formula III after appropriate activation of the carboxylic acid function as known for preparation of analogues 5-membered lactams. Both compounds of formula III or XII can be reacted with compound of formula V providing compound of formula IV which is known intermediate in the synthesis of the final compound of formula I (Aliskiren) as reported in U.S. Patent Appl. 61/279,995 from Oct. 29, 2009, WO 2007/045420 from Apr. 26, 2007, WO 2008/119804 from Oct. 9, 2008 and WO 2008/155338 from Dec. 24, 2008.

As a further embodiment of the invention, the starting compounds of formula II and IIa can be prepared in many ways, preferably as shown in Scheme 2:

Deprotonation of the compound of formula VIII (isobutyric acid or derivatives thereof), wherein X is the same as defined for compound of formula II, with a strong inorganic or organic base, preferably lithium amides as LDA or LiHMDS etc., and subsequent alkylation of the enolate with compound of formula VII, wherein Y is any possible leaving group, preferably cis-1,4-dichloro- or 1,4-dibromo-but-2-ene or Mesylate, derived from corresponding cis-but-2-ene-1,4-diol, leads to the compound of formula II. It is important that cis-compound of formula VII is isomerically pure in order to obtain pure cis-configurated compound of formula II. As an alternative, instead of compound of formula VIII isopropylmalonate or isopropyl malodinitrile can be used which can be more easily deprotonated, preferably with e.g. sodium hydride in THF or even in aqueous sodium hydroxide solution under phase transfer conditions (PTC), and then alkylated in the same way. In this case the alkylation product has to be subjected to a decarboxylation step either on a stage of free carboxylic acid or ester thereof as reported by e.g. Krapcho in Tetrahedron Letters 1967, 8, 215.

Enantiomerically pure cis-compound of formula II with configuration as shown in Schemes 1 and 2 can be prepared by a classical racemate resolution as e.g. reported in WO 2007/048620 from Mai 3, 2007. In another and more preferred approach the enantiomerically pure compound of formula VIII, wherein X represents —NR⁸R⁹ and R⁸ and R⁹ form together with N a 5- or 6-membered heterocyclic ring which may contain one or more heteroatoms selected from N or O and, which can be unsubstituted or substituted, containing also a chiral center, preferably such as e.g. 4(R)- or 4(S)-benzyl-oxazolidin-2-one-3-yl (Evans auxiliary), can be deprotonated with a strong organic base as LDA or LiHMDS and the enolate alkylated with cis-1,4-dibromobut-2-ene.

The enantiomerically pure, compound of formula II with specifically cis-configurated double bond has not yet been reported: Previous patents claimed either corresponding trans-isomer only or in US patent WO 2008/155338 only a stereoisomeric cis/trans-mixture containing less than 10% of the cis-isomer because the disclosed preparation method (cross-metathesis), as shown in experimental part, does not allow stereoselective synthesis of cis-configurated compound of formula II.

As alternative cis-configurated compound of formula II can also be prepared by analogous method as reported above using Evan chiral auxiliary wherein, instead of cis-1,4-dibromo-but-2-ene, compound of formula VIIla, preferably 1,4-dibromo-but-2-yne or Mesylate or Tosylate derived from but-2-yne-1,4-diol, has been used and after alkylation step, the triple bond in compound of formula XIII then subjected to partial hydrogenation, preferably with Lindlar catalyst (Scheme 2).

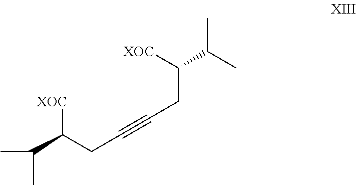

As a further embodiment of the invention a chiral acetylene of formula IX, wherein Z is halogen, preferably chlorine or bromine, is reacting with alkali or earth alkali metal, preferably lithium, magnesium or Sn, Al, Zr or In, providing corresponding organometallic compound which in situ can be subjected in the presence of catalytic amount of transition metals and phospine ligands, preferably Palladium complexes as e.g. Pd₂(dba)₃.CHCl₃ and (o-tolyl)₃P, reaction with compound of formula VII, wherein Y is —OC(O)alkyl or aryl or —OC(O)Oalkyl, preferably —OAc or —OC(O)OMe, providing the enantiomerically pure cis-configurated compound of formula IIa. Reaction conditions and solvents can be used as reported in Bull. Korean Chem. Soc. 2005, 26(1), 157, Tetrahedron Letters 1980, 21, 2599, Tetrahedron 1985, 41, 5779 and Chem. Rev. 2003, 103, 2921 and ibid. 1996, 96, 395. If enantiomerically pure compound of formula IX is used, the final product of formula IIa is also enantiomerically pure because during the Pd-catalyzed reaction the chiral centers remain untouched. Chiral compound of formula IX can be prepared from inexpensive known chiral alcohol of formula IX, wherein Z is hydroxy, —OMesyl, —OTosyl or —OSO₂CF₃, by enzymatic resolution of the corresponding racemate. The enantiomerically pure compound of formula IIa can be selectively oxidized with e.g. periodate in the presence of various transition metal catalysts such as RuO₂ giving the compound of formula II which can be used as discussed above.

When referring to compounds described in the present invention, it is understood that references are also being made to salts thereof.

The example are provided to illustrate particular aspects of the disclosure and do not limit the scope of the present invention as defined by the claims.

### EXAMPLES

Determination of optical purity was carried out with HPLC using chiral columns as Chiralcel OJ-H, Chiralpak AS-H or Chiralpak AD-H from Daicel Chem. Ind. In some cases the optical purity was also determined with NMR-Spectroscopy using chiral Eu-shift reagent. If not mentioned otherwise, all evaporation are performed under reduced pressure, preferably between 5-50 Torr in some case even under high vacuum. The structure of final products, intermediates and starting materials is confirmed by standard analytical methods, e.g. spectroscopic characteristics as MS or NMR or IR. Abbreviation used are those conventional in the art.

### Preparation of Compound (Ia, Aliskiren) from Compound (VIIIa, 3-isovaleroyl-4(S)-benzyloxazolidin-2-one)

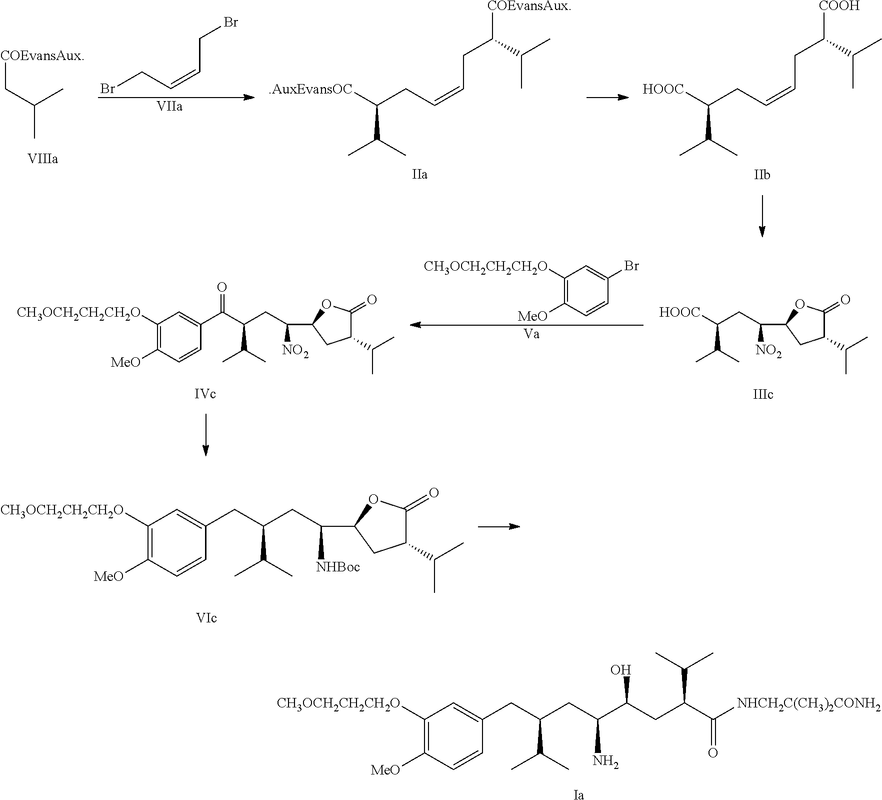

### Example 1

### Preparation of cis-2(S),7(S)-diisopropyl-octe-4-enedioic acid (IIb) from VIIIa

To a solution of 4(S)-benzyl-3-isovaleroyl-oxazolidin-2-one (12 g, THL 2000, 41, 10085), dissolved in THF (80 ml), under inert atmosphere cooled to −70° C. 1M-solution of lithium hexamethyldisilazide in toluene (LiHMDS, 50 ml) was slowly added dropwise under stirring at −70° C. within a period of ca. 1 hr. After stirring at the same temperature for 1 hr the reaction mixture was wormed to 0° C., then again cooled down to −70° C. and cis-1,4-dibromo-but-2-ene (4.5 g) in THF (10 ml) was slowly added, the reaction mixture shortly stirred at −70° C., then warmed to it and stirred for 7 hrs and finally poured on mixture of ice water and saturated sodium chloride solution (400 ml, 1:1). The aqueous phase was extracted 3 times with ethylacetate (3×200 ml), the combined organic phases washed once with saturated sodium bicarbonate solution (200 ml), dried with sodium sulphate, filtered and the filtrate evaporated under vacuum providing compound (IIa) as cis-2(S),7(S)-diisopropyl-oct-4-enedioic acid [bis((4(S)-benzyl-oxazolidin-2-one)]amide as a single diastereomer: crude 9.2 g (77% isolated yield) as a yellow semi crystalline oil. To stirred solution of the crude compound (IIa) (9.2 g), dissolved in a mixture of THF (100 ml) and water (30 ml), at 0° C. 35% aqueous hydrogen peroxide (30 ml) followed by 5 M aqueous solution of LiOH (70 ml) were added. After stirring for 1 hr at 0° C. the solution was warmed to rt and stirred over night. After addition of 0.5 M aqueous solution of Na₂SO₃ (70 ml) and water (70 ml) the aqueous phase was washed 3 times with MTBE to recover the chiral auxiliary. The aqueous phase was then acidified with conc.-HCl to pH 1, extracted 3 times with MTBE (3×200 ml), the combined organic phases dried over MgSO₄, filtered and the filtrate concentrated under reduced pressure providing the title compound (IIb) (cis-2(S),7(S)-2,7-diisopropyloct-4-enedioic acid) as a single diastereomer as white crystals crude 3.9 g (95% isolated yield): Anal. calculated for C₁₄H₂₄O₄: C, 65.60; H, 9.44; O 24.97. Found: C, 65.53; H, 9.38; O 24.88.

### Example 2

### Preparation of Compound (IIIc) from Compound (IIb) Using AcONO₂

90% Nitric acid (10 g) was very slowly dropped into stirred and ice bath cooled acetic anhydride (60 g) at such a rate that the reaction temperature was maintained at it and then the solution then stirred for 20 min. This was followed by dropwise addition of cis-2(S),7(S)-2,7-diisopropyloct-4-enedioic acid (IIa, 25 g), dissolved in acetic acid (10 ml), within ca. 1 hr and continuous cooling with ice bath in order to maintain the temperature at rt. After stirring for 1 hr at rt, the reaction mixture was poured carefully on a mixture of ice and water (ca. 200 ml), the aqueous phase extracted 3 times with methylenechloride

(3×200 ml), the combined organic phases washed twice with water (2×200 ml), then dried over MgSO4, filtered and evaporated under vacuum. The crude residue was dried on high vacuum to remove last traces of acetic acid and anhydride providing the title compound (IIIc) with a structure as indicated in the Scheme above as brown semi crystalline oil: 25.3 g (84% isolated yield). A small sample was purified by a column chromatography on silicagel, eluens: hexane/ethyl acetate (10:1): Anal. calculated for C₁₄H₂₃NO₆: C, 55.80; H, 7.69; N, 4.65; O 31.86. Found: C, 55.51; H, 9.50; N, 4.92; O 31.79.

### Example 3

### Preparation of Compound (IIIc) from Compound (IIb) using CAN ((NH₄)₂Ce(NO₃)₅.4H₂O)

A mixture of cis-2(S),7(S)-2,7-diisopropyloct-4-enedioic acid (IIa, 2.5 g) and CAN (1.7 g) in acetic anhydride (8 ml) was stirred at rt for 10 hrs, then the reaction mixture poured on crushed ice (100 g) and the aqueous phase extracted 3 times with ethylacetate (3×100 ml). The combined organic phases were washed once with water (100 ml), dried over MgSO4, filtered and evaporated under vacuum. The crude residue was finally dried on high vacuum to remove last traces of acetic acid/anhydride providing the title compound (111c) with a structure as indicated in the Scheme above as brown oil: 1.7 g (57% isolated yield) with analytical data identical with the product prepared as given in Example 2.

### Example 4

### Preparation of Compound (IVc) from Compound (IIIc)

a) Activation of compound (IIIc) as acid chloride: To a compound (IIIc, 30 g) dissolved in methylenechloride (100 ml) was added in inert atmosphere and under stirring at rt thionylchloride (15 g) and the mixture stirred for ca. 3 hrs, then evaporated under reduced pressure (2 Torr) to dryness: The residue was then dissolved in dry THF (100 ml) and used in situ as shown below in section c).

b) Preparation of organomagnesium ate complex from compound (Va).: To 1M-solution of butylmagnesium bromide in THF (120 ml) in THF (100 ml) 1.56M-hexane solution of butyllithium (78 ml) was added at 0° C. and the mixture stirred for 10 min. A solution of bromide (Va, 27 g), dissolved in THF (100 ml), was slowly introduced dropwise and after stirring for 1 hr at 0° C. the reaction mixture was cooled to −78° C.

c) Reaction of magnesium ate complex as prepared above in section b) with acid chloride prepared in section a): In inert atmosphere under stirring to solution of acid chloride (IIIc, prepared in section a)) cooled to −50° C., solution of the magnesium ate complex (prepared in section b)), cooled to −78° C., was slowly added over a time of ca. 1 hr, the mixture stirred at the same temperature for 4 hrs and then poured slowly within ca. 20 min on a mixture of toluene (150 ml) and 10% aqueous solution of citric acid (350 ml) (exothermic reaction!). The organic phase was separated, the water phase extracted 3 times with toluene (3×100 ml), the combined organic phases washed once with 10% aqueous 10% solution of citric acid (150 ml), then with water (2×100 ml), dried with MgSO₄, filtered and the filtrate evaporated under reduced pressure to yield crude compound (IVc) as yellow oil: 39.5 g (82% isolated yield). A small sample of crude (IVc) was purified by a column chromatography on silicagel, eluens: hexane/toluene (10:1): Anal. calculated for C₂₅H₃₇NO₈: C, 62.61; H, 7.78; N, 2.92; O 26.69. Found: C, 62.69; H, 7.71; N, 2.81; O 26.59.

### Example 5

### Preparation of Compound (Ia) from Compound (Ivc) Via Compound (VIc)

Crude lactone (IVc) from the above experiment (Example 4) (5 g) was dissolved in THF (40 ml), 10% Pd—C (400 mg), N,N-dimethyl aminopyride (0.1 g), triethylamine (4 g) and di-tert-butyldicarbonate (3 g) were added at rt and the mixture hydrogenated under slightly elevated pressure under intensive stirring for 24 hrs to achieve complete reduction and BOC-protection of the 5-amino group. After careful acidification of the reaction mixture with glacial acetic acid, the mixture was poured on toluene/water mixture (300 ml 1:1) and the organic phase separated, dried over MgSO₄, filtered and the filtrate evaporated under reduced pressure: 4.9 g (90% isolated yield) of BOC-protected lactone (VIc) [(1S,3S)-1-((2S,4S)-4-isopropyl-5-oxo-tetrahydro-furan-2-yl)-3-[4-methoxy-3-(3-methoxypropoxy)-benzyl]-4-methyl-pentyl]-carbamic acid tert.-butyl ester. The analytical data were identical as reported e.g. in WO2006/024501, p. 58).

A solution of BOC-protected lactone (VIc) (4.9 g), 3-amino-2,2-dimethyl propionamide (1.7 g) and 2-hydroxypyridine (1 g) in TBME (20 ml), containing triethylamine (0.25 ml), was stirred for 18 hrs at 80° C., then cooled to rt and diluted with toluene (20 ml) and washed with 10% aqueous sodium hydrogen sulphate solution (100 ml). The organic phase was separated, washed once with water (50 ml), dried with magnesium suphate, filtrated and evaporated under reduced pressure to give a yellow oil which was suspended in hexane (100 ml), slurry stirred a few min, filtrated and the filtrate evaporated under reduced pressure providing a foam of BOC-protected derivative of Aliskiren (Ia): 4.5 g. The crude BOC-protected Aliskiren (4.5 g) was dissolved in a solution of trifluoro acetic acid and dichloromethane (30 ml, 1:5) at rt, stirred for 2 hrs and then pH adjusted to 10 with 37% sodium hydroxide solution. The aqueous phase was extracted 3 times with dichloromethane (3×100 ml), dried with magnesium sulphate, filtrated and the filtrate evaporated under reduced pressure providing yellow oil of Aliskiren (Ia): 3.9 g: The analytical date were identical with reported in EP 0678 503, example 137.

### Preparation of Compound (Ia, Aliskiren) from Compound (IIIc) via Grignard Route

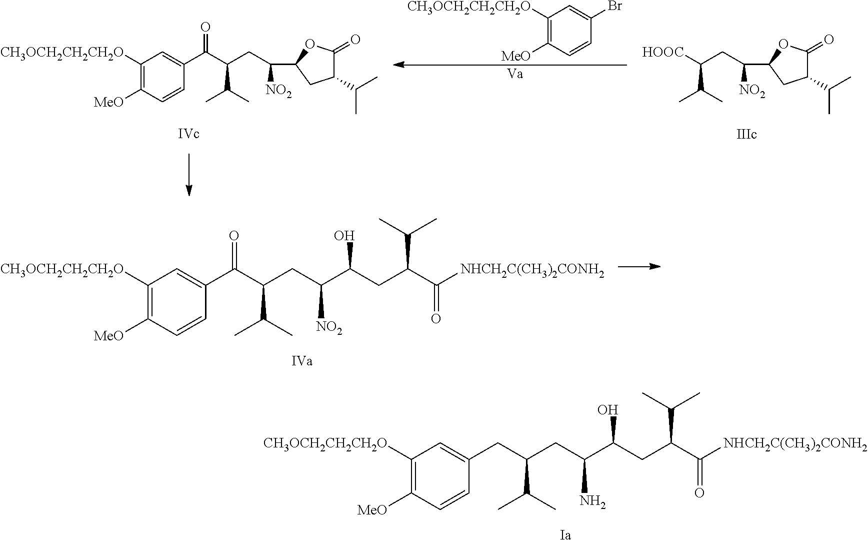

### Example 6

### Preparation of Compound (IVc) from Compound (IIIc)

a) Activation of compound (IIIc) as acid chloride: To a compound (IIIc, 30 g) dissolved in methylenechloride (100 ml), was added in inert atmosphere and under stirring at rt thionylchloride (15 g) and the mixture stirred for ca. 3 hrs, then evaporated under reduced pressure (2 Torr) to dryness: The residue was then dissolved in dry THF (100 ml) and used in situ as shown below.

b) Preparation of the Grignard reagent (Va): Several crystals of iodine were added to a suspension of magnesium turnings (6 g) in THF (150 ml) and the mixture was stirred at rt under nitrogen for ca. 3 hrs, then 10 drops of 1,2-dibromo butane were added and the mixture stirred for another 30 min. To this slurry compound (Va) (28 g), dissolved in dry THF (50 ml), was slowly added under stirring that the reaction mixture started to reflux. When the addition was complete the reaction mixture was maintained under reflux for 1 hr.

c) Reaction of compound prepared in section a) with Grignard reagent prepared in section b): Grignard reagent from section b was cooled to it and added dropwise within a period of ca. 1 hr to a solution of acid chloride prepared from compound (IIIc) from in section a), dissolved in dry THF (150 ml), and cooled to −78° C. The slurry was stirred at −78° C. for 4 hrs, and then poured slowly within ca. 20 min on a mixture of toluene (250 ml) and 10% aqueous solution of citric acid (250 ml) (exothermic reaction!). The organic phase was separated, the water phase extracted 3 times with toluene (3×100 ml), the combined organic phases washed once with 10% aqueous solution of citric acid (150 ml), then with water (2×100 ml), dried with MgSO₄, filtered, the filtrate evaporated under reduced pressure to yield crude compound (IVc) as yellow oil: 35. g (73% isolated yield).

### Example 7

Preparation Compound (Ic) from Compound (IVc) via Compound (IVa)

At rt under intensive stirring to a solution of compound (IVc), prepared in example 6, (4.8 g) in methanol (40 ml) LiOH (0.3 g) was added and stirring was continued for 45 min. After addition of acetic acid (1.5 ml) the crude mixture was poured on aqueous saturated NaCl solution (100 ml) and the aqueous phase extracted 5 times with TBME (5×80 ml), the combined organic phases dried over MgSO4, filtered, the filtrate evaporated under vacuum providing crude carboxylic acid which was immediately taken with THF (40 ml) and after addition of DCC (3 g), N-hydroxy succinimide (1.2 g) and 3-amino-2,2-dimethyl propionamide (3 g), stirred at rt over night. After filtration of the slurry, the filtrate was diluted with toluene (100 ml) and washed with aqueous saturated NaCl solution (100 ml). The organic phase was separated, washed once with water (50 ml), dried with magnesium suphate, filtrated and evaporated under reduced pressure to give a compound (IVa) as a yellow oil.

The residue of crude (IVa) was then dissolved in acetic acid (50 ml) and after addition of 10% Pd—C under intensive stirring the slurry hydrogenated under normal pressure at rt for ca. 3 hrs. The reaction mixture was then filtered to remove the catalyst, poured on water (300 ml) and pH adjusted with 37% sodium hydroxide solution to 10. The final product, Aliskiren was then extracted 4 times with dichloromethane (4×100 ml), the organic phase evaporated under reduced pressure providing crude Aliskiren (Ia): 3.0 g with identical analytical data as reported e.g. in EP 0678503 p. 74, example 137: MS (M⁺ 552), Rf 0.33 on silicagel eluens: dichloromethane/methanol=8:2.

From the free base (Ia) the hemifumarate salt was prepared as described in U.S. Pat. No. 6,730,798 example J1.

### Example 8

### Preparation of Compound (XIIa) from Compound (IIb)

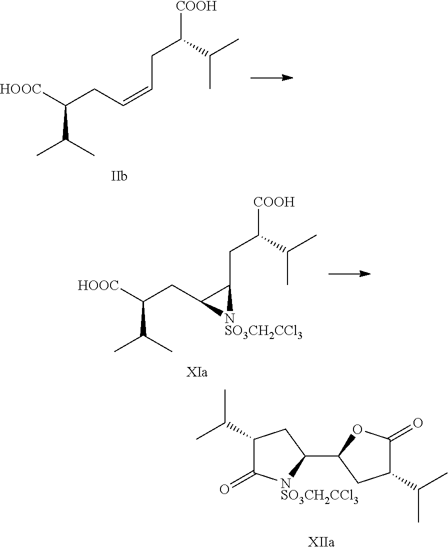

Preparation of Compound (XIa) from Compound (IIb):

All reagents used in this experiment have been prepared according to J. Amer. Chem. Soc. 2002, 124(46), 13673.

To a solution of NH₂SO₃CH₂CCl₃ (23 g) in toluene (100 ml) were added sequentially cis-2(S),7(S)-2,7-diisopropyloct-4-enedioic acid (IIb, 25 g), MgO (9 g) and Rh₂(tfacam)₄ (50 mg) and the resulting purple mixture cooled to 0° C. before PhI(OAc)₂ (42 g) was added. Under stirring and ice cooling the reaction mixture (exothermic!) was allowed to warm to rt for ca. 8 hrs, then carefully acidified with acetic acid (ca. 60 ml), diluted with methylenechloride (200 ml), filtered through Celite (30 g), the filter cake washed with more methylenechloride (200 ml) and the combined filtrates concentrated under reduced pressure providing aziridine (XIa) as slightly brown semi crystalline oil: 45.3 g (92% isolated yield) which was directly used for the next step.

Preparation of Bicyclic Compound (XIIa) from Aziridine (XIa):

Crude aziridine (XIa, 45.3 g) was dissolved in methylenechloride (200 ml) and the solution after cooling to 0° C. under stirring slowly treated with trifluoro acetic acid (30 ml) and kept at 0° C. for ca. 30 min, then allowed to warm to it for another 50 min. and then evaporated under reduced pressure to dryness. The residue was dissolved in methylenechloride (200 ml) and after addition of DCC (25 g), the slurry stirred at it for 5 hrs then filtered, the filtrate poured on mixture of ice and water (500 ml), the aqueous phase extracted 3 times with methylenechloride (3×200 ml), the combined organic phases washed once with water (200 ml) and evaporated under reduced pressure providing the title bicyclic compound (XIla) as brown oil: 39.5 g (87% isolated yield).

Small sample of the crude material was purified by column chromatography on silicagel, eluens: hexane/ethyl acetate (10:2): Anal. calculated for C₁₆H₂₄Cl₃NO₆S: C, 41.35; H, 5.20; Cl 22.88; N 3.01; O 20.65; S 6.90. Found: C, 41.33; H, 5.12; Cl 22.92; N 3.09; O 20.72; S 2.89.

### Example 9

### Preparation of Compound (XIIb) from Compound (IIb)

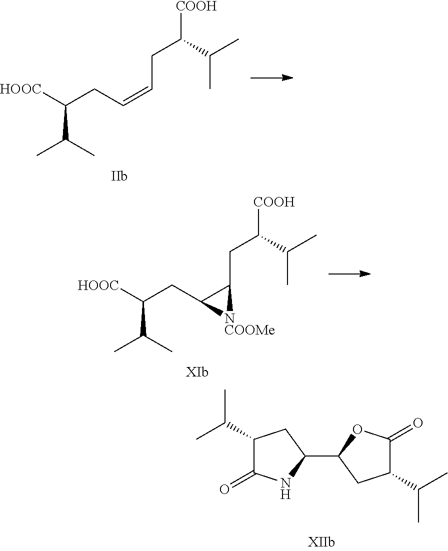

Preparation of Compound (XIb) from Compound (IIb)

Under inert atmosphere to a stirred slurry of 2(S),7(S)-2,7-diisopropyloct-4-enedioic acid (IIa, 2.5 g) in dichloroethane (30 ml), at 70° C. slowly azido formate methylester (1.5 g) was dropwise added within ca. 30 min and the mixture refluxed for 30 min. In case that all starting material (IIb) has not been consumed additional azido formate methylester was added at the same temperature. After cooling to rt the reaction mixture was evaporated to dryness under reduced pressure providing aziridine (XIb) as a brown oil: 3.0 g (91% isolated yield) which was directly used for the next step.

Preparation of Bicyclic Compound (XIIb) from Aziridine (XIb):

Crude aziridine (XIb, 3 g) from the above experiment was dissolved in methanol and after addition of KOH (3 g) the solution stirred for 2 hrs and then evaporated to dryness under reduced pressure. The residue was taken with methylenechloride (100 ml) under intensive stirring cooled to 0° C. After slow addition of trifluoro acetic acid (5 ml) and trifluoro methane sulphonic acid (50 mg) at 0° C. the reaction slurry was stirred for ca. 30 min, then evaporated under reduced pressure to dryness, the residue suspended again in methylenechloride (100 ml), the slurry poured on water (50 ml), the aqueous phase 5 times extracted with methylenechloride (5×70 ml), the combined organic phases evaporated under reduced pressure. The residue was dissolved in methylenechloride (50 ml) and after addition of DCC (2.5 g) a few drops of triethylamine have been added and the reaction mixture stirred for ca. 5 hrs. After addition of acetic acid (2 ml) the reaction slurry was filtered, the filtrate then poured on buffer solution pH 7 (100 ml), the aqueous phase 3 times extracted with methylenechloride (3×70 ml), the combined organic phases washed once with water (50 ml) and evaporated under reduced pressure providing the title bicyclic compound (XIIb) as brown oil: 1.7 g (73% isolated yield). Small sample of the crude material was purified by column chromatography on silicagel, eluens: hexane/ethyl acetate (10:1): The analytical data of the bicyclic compound (XIIb) were identical as reported in WO 2007/045420 on page 61.

1. A compound of a general formula III, 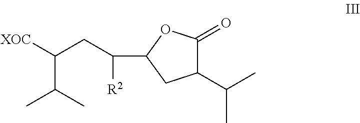
wherein R² represents NO₂ and
X represents —OH, linear or brunched C₁₋₆-alkyloxy, aryloxy, alkylaryloxy, arylalkyloxy, trialkylsilyloxy or alkylarylsilyloxy, preferably —OH, —OMe, halogen, preferably chlorine or bromine,
—OC(O)R⁷ or —OC(O)OR⁷, wherein R⁷ is linear or brunched C₁₋₆-alkyl, arylalkyl, preferably methyl, ethyl, tert.-butyl or benzyl,
—NR⁸R⁹, wherein R⁸ and R⁹ are independently from each other hydrogen, lower alkyl, alkylaryl, arylalkyl, preferably —NH₂, —NMe₂, or in particular R⁸ and R⁹ can form together with N a 5- or 6-membered heterocyclic ring which may contain one or more heteroatoms selected from N or O and, which can be unsubstituted or substituted, preferably 4-alkyl-oxazolidin-2-one-3-yl, containing also a chiral center as 4(R)- or 4(S)-benzyl-oxazolidin-2-one-3-yl, or
—NR¹⁰OR¹¹, wherein R¹⁰ and R¹¹ are independently from each other lower alkyl, alkylaryl, arylalkyl, preferably —NMeOMe, or in particular R¹⁰ and R¹¹ can form together with N and O a 5- or 6-membered heterocyclic ring which may contain one or more heteroatoms selected from N or O and, which can be unsubstituted or substituted, containing also a chiral center and a salt thereof.

2. The compound according to claim 1, having the configuration as given in the formula

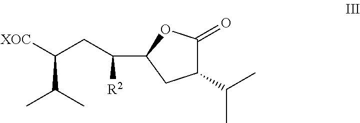

3. A compound of a general formula IIIa, 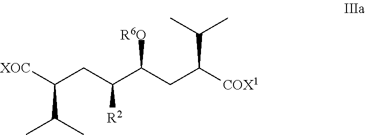
wherein R² is NO₂,
X and X¹ are independent from each other the same as defined for X in the compound of formula III in claim 1,
R⁶ represents hydrogen, alkyl, aryl, alkylaryl, arylalkyl, trialkylsilyl, alkylarylsilyl, with heteroatom(s) substituted alkyl, aryl, arylalkyl, preferably benzyl, mono-, di- or tri-methoxybenzyl, or other O-protective group, in particular one which together with O forms an ester or carbonate as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably acetyl, formyl, —C(O)O-benzyl (Cbz) or —C(O)Otert.-butyl (BOC);
and a salt thereof,
as well as all possible stereo isomers thereof as enantiomerically pure compound or racemate or mixtures thereof.

4. A compound of a general formula IV, 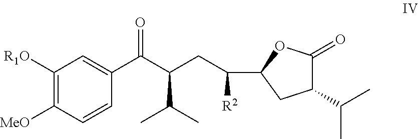
wherein R¹ represents hydrogen, linear or brunched C₁₋₆ alkyl, C₁₋₆ alkoxy-C₁₋₆ alkyl, aryl, alkylaryl, arylalkyl, preferably CH₃OCH₂CH₂CH₂—, acyl, formyl, trifluoracetyl, Mesyl, Tosyl, trifluoromethanesulfonyl, trialkylsilyl, and R² is NO₂ or NR⁴R¹², wherein R⁴ and R¹² are independently from each other arylalkyl, preferably benzyl, mono-, di- or trimethoxybenzyl, trialkylsilyl or other N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably formyl, acetyl, trifluoroacetyl, —C(O)Obenzyl (Cbz) or —C(O)Otert.-butyl (BOC), or Mesyl and Tosyl and a salt thereof, as well as all possible stereo isomers thereof as either enantiomerically pure compound or racemate or mixtures thereof.

5. A compound of general formula IVa, 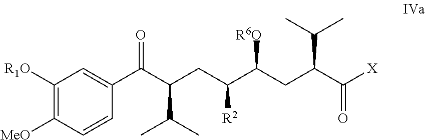
wherein R¹ and R² are the same as defined for the compound of formula IV in claim 4,
X is the same as defined for the compound of formula III in claim 1,
R⁶ is the same as defined for the compound of formula IIIa in claim 3 and a salt thereof, as well as all possible stereo isomers thereof as enantiomerically pure compound or racemate or mixtures thereof.

6. A compound of general formula IVb, 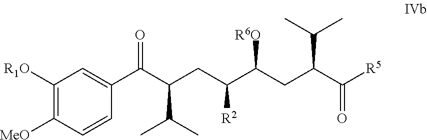
wherein R¹ and R² are the same as defined for compound of formula IV in claim 4,
R⁵ is hydroxy, linear or brunched C₁₋₆ alkyloxy, aryloxy, alkylaryloxy, arylalkyloxy, trialkylsilyloxy or alkylarylsilyloxy, halogen, preferably chlorine or bromine, —NH₂, —NMe₂, preferably —NHCH₂C(CH₃)₂CONH₂ and
R⁶ is the same as defined for compound of formula IIIa in claim 3
and a salt thereof, as well as all possible stereo isomers thereof as enantiomerically pure compound or racemate or mixtures thereof.

7. A compound of a general formula II, 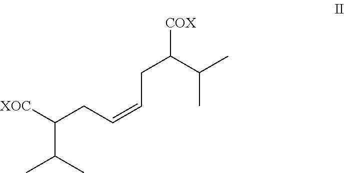
wherein X represents —OH, linear or brunched C₁₋₆-alkyloxy, aryloxy, alkylaryloxy, arylalkyloxy, trialkylsilyloxy or alkylarylsilyloxy, preferably —OH and OMe, or
—OC(O)R⁷ or —OC(O)OR⁷, wherein R⁷ is linear or brunched C₁₋₆-alkyl, arylalkyl, preferably methyl, tert.-butyl, benzyl, or
halogen, preferably chlorine or bromine, or
—NR⁸R⁹, wherein R⁸ and R⁹ are independently from each other hydrogen, lower alkyl, alkylaryl, arylalkyl, preferably —NH₂, —NMe₂, —Ndibenzyl, or in particular R⁸ and R⁹ can form together with N a 5- or 6-membered heterocyclic ring which may contain one or more heteroatoms selected from N or O and, which can be unsubstituted or substituted, preferably 4-alkyl-oxazolidin-2-one-3-yl, containing also a chiral center as 4(R)- or 4(S)-benzyl-oxazolidin-2-one-3-yl, or
—NR¹⁰OR¹¹, wherein R¹⁰ and R¹¹ are independently from each other lower alkyl, alkylaryl, arylalkyl, preferably —NMeOMe, or in particular R¹⁰ and R¹¹ can form together with N and O a 5- or 6-membered heterocyclic ring which may contain one or more heteroatoms selected from N or O and, which can be unsubstituted or substituted, containing also a chiral center, and
wherein the double bond is specifically cis-configurated and a salt thereof.

8. The compound according to claim 7, having the configuration as given in the formula

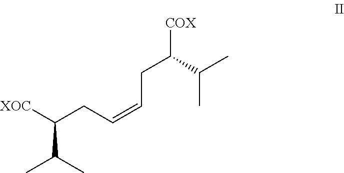

9. The compound of formula IIa, having the configuration as given in the formula

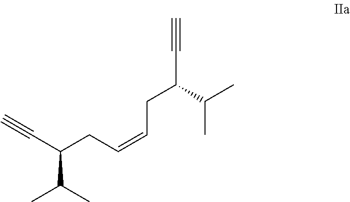

10. A compound of a general formula XI 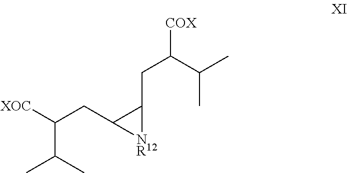
wherein X is OH, linear or brunched C₁₋₆-alkyloxy, aryloxy, alkylaryloxy, arylalkyloxy, trialkylsilyloxy or alkylarylsilyloxy, preferably —OH or —OMe, or
—OC(O)R⁷ or O C(O)OR⁷, wherein R⁷ is linear or brunched C₁₋₆-alkyl, arylalkyl, preferably methyl, tert-butyl, benzyl, or
halogen, preferably chlorine or bromine, or
—NR⁸R⁹, wherein R⁸ and R⁹ are independently from each other hydrogen, lower alkyl, alkylaryl, arylalkyl, preferably —NH₂, —NMe₂, —Ndibenzyl, or in particular R⁸ and R⁹ can form together with N a 5- or 6-membered heterocyclic ring which may contain one or more heteroatoms selected from N or O and, which can be unsubstituted or substituted, preferably 4-alkyl-oxazolidin-2-one-3-yl, containing also a chiral center as 4(R)- or 4(S)-benzyl-oxazolidin-2-one-3-yl, or —NR¹⁰OR¹¹, wherein R¹⁹ and R¹¹ are independently from each other lower alkyl, alkylaryl, arylalkyl, preferably —NMeOMe, or in particular R¹⁰ and R¹¹ can form together with N and O a 5- or 6-membered heterocyclic ring which may contain one or more heteroatoms selected from N or O and, which can be unsubstituted or substituted, containing also a chiral center and
R¹² is hydrogen, lower alkyl, arylalkyl, alkylaryl, —C(O)alkyl, —C(O)aryl, —C(O)alkylaryl, —C(O)arylalkyl, —C(O)Oalkyl, —C(O)Oalkylaryl, preferably formyl, acetyl, methyl, benzyl, —C(O)OMe or —C(O)Otert.-butyl, Mesyl and Tosyl
and a salt thereof.

11. The compound according to claim 10, having the configuration as given in the formula

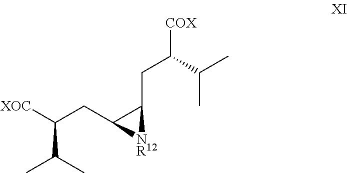

12. A process for the preparation of the compound of general formula I 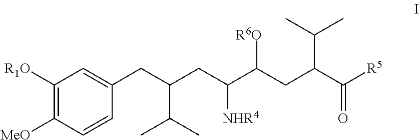
wherein
R¹ represents hydrogen, linear or brunched C₁₋₆ alkyl, C₁₋₆ alkoxy-C₁₋₆ alkyl, aryl, alkylaryl, arylalkyl, preferably CH₃OCH₂CH₂CH₂—, acyl, formyl, trifluoracetyl, Mesyl, Tosyl, trifluoromethanesulfonyl, trialkylsilyl,
R⁴ represents hydrogen, alkyl, aryl, alkylaryl, arylalkyl, hydroxy, alkoxy, aryloxy, arylalkyloxy, alkylaryloxy, trialkylsilyl, trialkylsilyloxy, with heteroatom(s) substituted alkyl, aryl, alkylaryl, arylalkyl, preferably benzyl, mono-, di- or tri-methoxybenzyl, or other N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —(O)COalkylaryl, —C(O)Oarylalkyl, preferably formyl, acetyl, trifluoroacetyl, —C(O)Obenzyl (Cbz) or —C(O)Otert.-butyl (BOC),
R⁵ represents hydroxy, linear or brunched C₁₋₆ alkyloxy, aryloxy, alkylaryloxy, arylalkyloxy, trialkylsilyloxy, halogen, preferably chlorine or bromine, —NH₂, —NMe₂ or preferably —NHCH₂C(CH₃)₂CONH₂,
R⁶ represents hydrogen, alkyl, aryl, alkylaryl, arylalkyl, trialkylsilyl, alkylarylsilyl, with heteroatom(s) substituted alkyl, aryl, alkylaryl, arylalkyl, preferably benzyl, mono-, di- or tri-methoxybenzyl or other O-protective group, in particular one which together with 0 forms an ester or carbonate, as —C(O)alkyl, —C(O)aryl, —C(O)alkylaryl, —C(O)arylalkyl, —C(O)Oalkyl, —C(O)Oaryl, —C(O)Oalkylaryl, —C(O)Oarylalkyl, preferably formyl, acetyl, —C(O)O-benzyl (Cbz) or —C(O)Otert.-butyl (BOC);
comprising following steps:
a) reaction of the compound of formula II 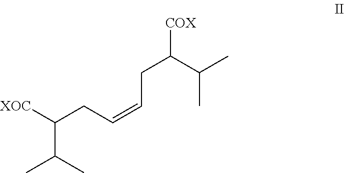
wherein X represents —OH, linear or brunched C₁₋₆-alkyloxy, aryloxy, alkylaryloxy, arylalkyloxy, trialkylsilyloxy or alkylarylsilyloxy, preferably —OH, OMe, or
—OC(O)R⁷ or —OC(O)OR⁷, wherein R⁷ is linear or brunched C₁₋₆-alkyl, arylalkyl, preferably methyl, tert.-butyl or benzyl, or
halogen, preferably chlorine or bromine, or
—NR⁸R⁹, wherein R⁸ and R⁹ are independently from each other hydrogen, lower alkyl, alkylaryl, arylalkyl, preferably —NH₂, —NMe₂, —Ndibenzyl, or in particular R⁸ and R⁹ can form together with N a 5- or 6-membered heterocyclic ring which may contain one or more heteroatoms selected from N or O and, which can be unsubstituted or substituted; preferably 4-alkyl-oxazolidin-2-one-3-yl, containing also a chiral center as 4(R)- or 4(S)-benzyl-oxazolidin-2-one-3-yl, or
—NR¹⁰OR¹¹, wherein R¹⁰ and R¹¹ are independently from each other lower alkyl, alkylaryl, arylalkyl, preferably —NMeOMe, or in particular R¹⁰ and R¹¹ can form together with N and O a 5- or 6-membered heterocyclic ring which may contain one or more heteroatoms selected from N or O and, which can be unsubstituted or substituted, containing also a chiral center, and
wherein the double bond is specifically cis-configurated,
with a “nitration agent” containing NO₂⁺-agent, preferably nitration agent defined as NO₂-Lvg, wherein Lvg is a leaving group, preferably NO₂OAc, NO₂BF₄, cerium(III)ammonium nitrate (CAN), providing compound of formula III, wherein X is are the same as defined for compound of formula II and R² is NO₂, 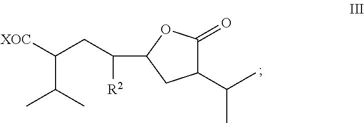
b) reaction of the compound of formula III with a compound of formula V, 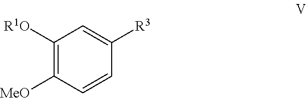
wherein R¹ is the same as defined for the compound of formula I and R³ is a metal containing group such as —Li, —Na, —Mghalide (Grignard reagent), Znhalide, Mnhalide, cuprate, —Cuhalide, —Cehalide, boronic acid as —B(OH)₂, preferably —Li or —MgBr, providing compound of formula IV, wherein R¹ is the same as defined for compound of formula I and R² is NO₂ 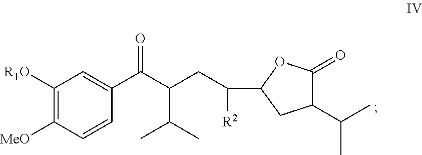
c) reaction of
i. either the compound of formula IV directly with R⁵—H, wherein R⁵ is the same as defined for the compound of formula I,
preferably —NHCH₂C(CH₃)₂CONH₂,
ii. or, after initial hydrolysis/opening of the lactone of formula IV, coupling of the free carboxylic acid with a peptide coupling reagent and then reaction with R⁵—H, wherein R⁵ is the same as defined for the compound of formula I, preferably —NHCH₂C(CH₃)₂CONH₂,
providing a compound of formula IVb, wherein R¹, R⁵ and R⁶ are the same as defined for compound of formula I and R² is NO₂, 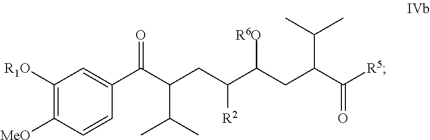
c) reduction or/and hydrogenation of C(8)-oxo and C(5)-nitro groups in the compound of formula IVb, either simultaneously or in separate steps, followed by subsequent protection or removal of protective group(s) according to as they are defined in the compound of formula I.

13. A process according to the claim 12, wherein the compound of formula I has the configuration as given 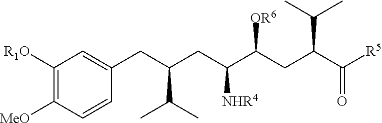
and the compounds of formulas II, III, IV and IVb have the configuration as defined in claims 8, 2, 4 and 6, respectively.

14. A process for the preparation of a compound of general formula I according to claim 12 or 13 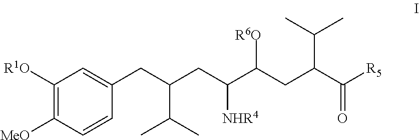
comprising following steps:
a) reaction of the compound of formula II 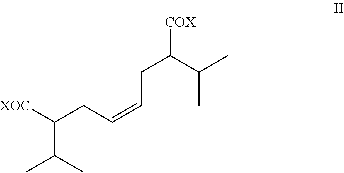
with a nitration agent containing NO₂⁺-agent providing compound of formula III 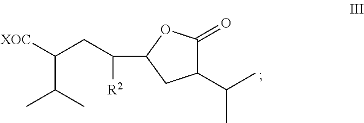
b) reaction of the compound of formula III with a compound of formula V, 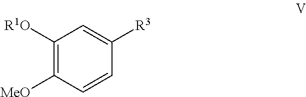
providing a compound of formula IV 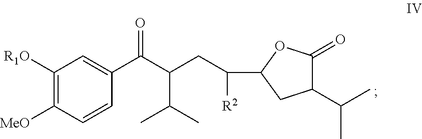
c) reduction or/and hydrogenation of C(8)-oxo and C(5)-nitro groups in the compound of formula IV, either simultaneously or in separate steps, providing after protection of the C(5)-amino group a compound of formula VI, or after opening of the lactone ring, a compound of formula VIa, wherein R¹, R⁴ and R⁶ are the same as defined for compound of formula I and X is the same as defined for compound of formula II; 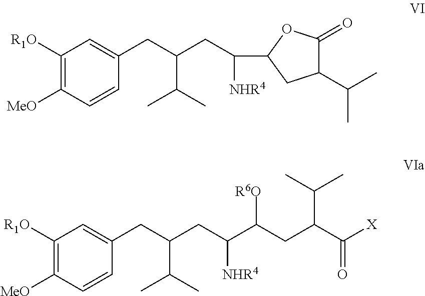
d) followed by
i. either reaction of the lactone of formula VI with R⁵—H, wherein R⁵ is the same as defined for the compound of formula I, preferably —NHCH₂C(CH₃)₂CONH₂
ii. or hydrolysis of the lactone of formula VI to the compound of formula
VIa and subsequent reaction of the free carboxylic acid with a peptide coupling reagent and reaction with R⁵—H, preferably NH₂CH₂C(CH₃)₂CONH₂,
and protection or removal of protective group(s) according to as they are defined in the compound of formula I.

15. A process according to the claim 14, wherein the compound of formula I has the configuration as given 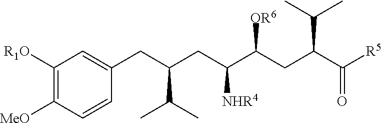
and the compounds of formulas II, III, IV, IVa, have the configuration as defined in claims 8, 2, 4 and 5, respectively and the compounds of formula VI and Vla as given

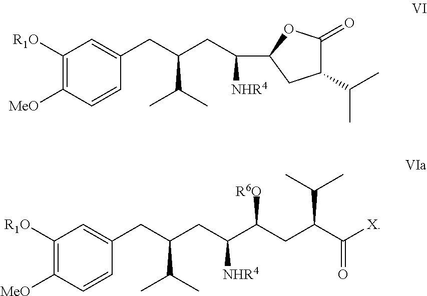

16. A process for the preparation of the compound of formula I as defined in claim 12 or 13 comprising following step:
a) reaction of the compound of formula II with nitration agent, preferably AcONO₂ or NO₂BF₄ or CAN providing a compound of formula III;
b) reaction of the compound of formula III with compound of formula V providing a compound of formula IV;
c) reaction of the compound of formula IV with NH₂CH₂C(CH₃)₂CONH₂;
d) reduction or heterogeneous hydrogenation of the compound of formula IVb.

17. A process for the preparation of compound of formula I according to claim 14 or 15 comprising following step:
a) reaction of the compound of formula II with nitration agent, preferably AcONO₂ or NO₂BF₄ or CAN, providing a compound of formula III;
b) reaction of compound of formula III with a compound of formula V providing a compound of formula IV;
c) reduction or/and hydrogenation of the compound of formula IV followed by protection of the C(5)-amino group providing compound of either formula VI or Vla;
d) reaction of compound of either formula VI or VIa with R⁵—H.

18. A process according to anyone of claims 12-17, wherein in compounds of formulas II, III, IIIa, IV, IVa, IVb, VI and VIa
R¹ represents CH₃OCH₂CH₂CH₂— or hydrogen,
R² represents NO₂,
R³ represents lithium, sodium, magnesium-chloride, bromide or —Mg ate complex,
R⁴ and R⁶ represents hydrogen, benzyl, dimethoxybenzyl, acetyl, formyl, —C(O)OMe, —C(O)Obenzyl (Cbz), —C(O)Otert.butyl (BOC), trifluoracetyl and benzyloxy,
R⁵ hydroxy, methoxy, ethoxy, benzyloxy and —NHCH₂C(CH₃)₂CONH₂,
R¹² represents hydrogen, benzyl, —C(O)OMe, —C(O)Obenzyl(Cbz), —C(O)Otert.butyl (BOC), acetyl, trifluoracetyl and —HNSO₃CH₂CCl₃ and
X. represents —OH, —OMe, —OEt, —OTMS, chlorine or bromine, —NH₂, —NMe₂, 4-alkyl-oxazolidin-2-one-3-yl, preferably 4(R) or 4(S)-benzyl-oxazolidin-2-one-3-yl and —NMeOMe.

19. A process for the preparation of a compound of formula XII 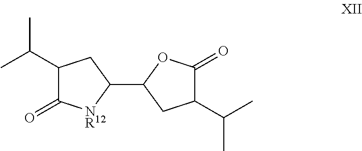
wherein R¹² is hydrogen, alkyl, aryl, alkylaryl, arylalkyl, trialkylsilyl, hydroxy, alkoxy, aryloxy, arylalkyloxy, alkylaryloxy, trialkylsilyloxy, with heteroatom(s) substituted alkyl, aryl, alkylaryl, arylalkyl, preferably benzyl, mono-, di- or tri-methoxybenzyl, or other N-protective group, in particular one which together with N forms an amide or carbamate as —C(O)alkyl, —C(O)aryl, —C(O)alkylaryl, —C(O)arylalkyl, —C(O)Oalkyl, —C(O)Oaryl, —(O)COalkylaryl, —C(O)Oarylalkyl, preferably formyl, acetyl, trifluoroacetyl, —C(O)Obenzyl (Cbz) or —C(O)Otert.-butyl (BOC), Mesyl, Tosyl and —SO₃CH₂CCl₃,
comprising following steps:
a) reaction of the compound of formula II 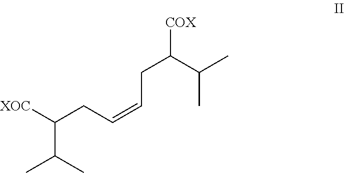
wherein X represents —OH, linear or brunched C₁₋₆-alkyloxy, aryloxy, alkylaryloxy, arylalkyloxy, trialkylsilyloxy or alkylarylsilyloxy, preferably —OH and OMe, or
halogen, preferably chlorine or bromine, or
—NR⁸R⁹, wherein R⁸ and R⁹ are independently from each other hydrogen, lower alkyl, alkylaryl, arylalkyl, preferably —NMe₂, —Ndibenzyl, or in particular R⁸ and R⁹ can form together with N a 5- or 6-membered heterocyclic ring which may contain one or more heteroatoms selected from N or O and, which can be unsubstituted or substituted, preferably 4-alkyl-oxazolidin-2-one-3-yl, containing also a chiral center as 4(R)- or 4(S)-benzyl-oxazolidin-2-one-3-yl, or
—NR¹⁰OR¹¹, wherein R¹⁰ and R¹¹ are independently from each other lower alkyl, alkylaryl, arylalkyl, preferably —NMeOMe, or in particular R¹⁰ and R¹¹ can form together with N and O a 5- or 6-membered heterocyclic ring which may contain one or more heteroatoms selected from N or O and, which can be unsubstituted or substituted, containing also a chiral center, and
wherein the double bond is specifically cis-configurated,
with a nitrene reactive intermediate prepared in situ
i. either by thermal decomposition of an alkyl or aryl or arylalkyl azide, preferably benzyl azide, or mesyl- or tosylazide or alkyl azidoformiate such as N₃COOMe or N₃COObenzyl or N₃COOtert.-butyl or an acyl azide, preferably AcN₃ or PhCON₃,
ii. or by base catalyzed decomposition of alkane or arene sulfonyl oxycarbamate such as alkyl- or benzyl- or tert.-butyl-OC(O)NHMesyl, preferably -Tosyl or —Nosyl or H₂NSO₃CH₂CCl₃,
in an inert organic solvent, preferably chlorinated hydrocarbons or THF, under room or elevated temperature,
followed by protection or re-protection or removal of protective group(s) X and/or R¹² according to as they are defined for the compound of formula XI, providing the compound of formula XI 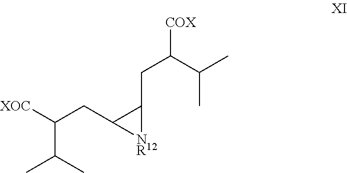
wherein X is the same as defined for compound of formula II and R¹² is the same as defined for compound of formula XII,
b) acid and/or base and/or metal catalyzed rearrangement of the aziridine of formula XI, formation of 5-membered lactone and subsequent ring closure with formation of 5-membered lactam ring.

20. A process according to the claim 19, wherein the compounds of formula II, XI and XII have the configurations as given [chemical structure]
